SEQUENCE LISTING (10 sequences)

SEQ ID NO: 1 - DNA, 13 bp, Homo sapiens
gcctttctcttcg

SEQ ID NO: 2 - DNA, 20 bp, Homo sapiens
tgtgacctctgctggttctg

SEQ ID NO: 3 - DNA, 22 bp, Homo sapiens
ttctccagcgacaaggctaagg

SEQ ID NO: 4 - DNA, 22 bp, Homo sapiens
ctccaacatccaatctgtcccg

SEQ ID NO: 5 - DNA, 20 bp, Homo sapiens
gcttgtgattgagaaccagg

SEQ ID NO: 6 - DNA, 22 bp, Homo sapiens
gaaagggaggctggtaaggaac

SEQ ID NO: 7 - DNA, 20 bp, Homo sapiens
gcctctccctagctttggtt

SEQ ID NO: 8 - DNA, 20 bp, Homo sapiens
ggtctgttaaggtgggcaga

SEQ ID NO: 9 - DNA, 20 bp, Homo sapiens
cacagtccatgccatcactg

SEQ ID NO: 10 - DNA, 20 bp, Homo sapiens
tactccttggaggccatgtg

(19) United States
(12) Patent Application Publication
(10) Pub. No.: US 2018/0360799 A1
(43) Pub. Date: Dec. 20, 2018

(54) Nitrofuran Derivatives That Induce Apoptosis in Breast Cancer Cells by Activating Protein Expression

(71) Applicant: Oklahoma Medical Research Foundation, Oklahoma City, OK (US)

(72) Inventor: Weidong Wang, Edmong, OK (US)

(73) Assignee: Oklahoma Medical Research Foundation, Oklahoma City, OK (US)

(21) Appl. No.: 15/735,700

(22) PCT Filed: May 6, 2016
(86) PCT No.: PCT/US16/31121

§ 371 Date: Dec. 12, 2017

Related U.S. Application Data
(60) Provisional application No. 62/158,924, filed on May 8, 2015.

(51) Int. Cl. A61K 31/345; A61K 31/4525; A61K 31/496; A61K 31/5377; A61P 35/00

(52) U.S. Cl. CPC A61K 31/345; A61K 31/4525; A61P 35/00; A61K 31/5377; A61K 31/496

(57) ABSTRACT

The present invention provides 5-nitrofuran-2-amide derivatives and methods of using the same in the treatment of cancer and induction of apoptosis by activating C/EBP-homologous protein expression.

### CROSS-REFERENCE TO RELATED APPLICATIONS

This application claims priority to, and is the National Stage of International Application No. PCT/US2016/031121 filed May 6, 2016 and claims the priority of U.S. Provisional Patent Application Ser. No. 62/158,924, filed on May 8, 2015, the contents of which are incorporated by reference herein in their entirety.

### TECHNICAL FIELD OF THE INVENTION

The present invention relates generally to methods and compositions used in the treatment of cancer and more specifically 5-nitrofuran-2-amide derivatives used in the treatment of triple negative breast cancer cells by inducing apoptosis by activating C/EBP-homologous protein expression.

### BACKGROUND OF THE INVENTION

Without limiting the scope of the invention, its background is described in connection with treating triple negative breast cancer cells by inducing apoptosis through activation of C/EBP-homologous protein expression using 5-nitrofuran-2-amide derivatives.

The endoplasmic reticulum is the major cellular organelle responsible for protein folding and secretion, calcium storage, calcium release, and lipid biogenesis, and disturbance can lead to the accumulation of misfolded or unfolded proteins. In response, the endoplasmic reticulum cells activate the unfolded protein response, a signaling pathway mediated by three ER transmembrane protein sensors: inositol-requiring enzyme 1α (IRE1α); protein kinase RNA-like ER kinase (PERK); and activating transcription factor 6 (ATF6); in an attempt to restore homeostasis.¹⁻³ The unfolded protein response is initiated as an adaptive mechanism to alleviate the accumulation of misfolded or unfolded proteins in the ER by altering protein translation, folding, and post-translational modifications.³⁻⁵ However, if these adaptive responses fail to re-establish homeostasis because endoplasmic reticulum stress is excessive or prolonged, a terminal unfolded protein response becomes activated and induces cell death.

Endoplasmic reticulum stress and unfolded protein response activation have been implicated in the pathogenesis of human cancers.⁶⁻⁹ Cancer cells utilize the adaptive branch of the unfolded protein response pathway to survive and progress in a stressful microenvironment. For example, the B-cell neoplasm multiple myeloma (MM) displays chronic endoplasmic reticulum stress, and is dependent on the adaptive Ire1α-X-box binding protein 1 (XBP1, an Ire1α substrate) branch of the unfolded protein response pathway for survival.⁹ Similarly, in triple negative breast cancer (TNBC), which is defined by the absence of the estrogen receptor, progesterone receptor, and human epidermal growth factor receptor-2 and is among the most aggressive and treatment-resistant forms of breast cancer¹⁰, XBP1 is highly activated and plays a pivotal role in the tumorigenicity and progression.⁸ Accordingly, inhibiting the adaptive Ire1α-XBP1 pathway has been proposed as a promising strategy for the development of anticancer therapy.^{8, 11, 12} Indeed, blockade of XBP1 activation by small molecule Ire1α inhibitors has been shown to cause significant growth inhibition of MM cells.^{12, 13} On the other hand, another recently proposed therapeutic rationale is to augment the terminal unfolded protein response in cancer cells whose adaptive unfolded protein response is active so as to tip the balance to apoptosis instead of survival.^{6, 14, 15}

The transcription factor C/EBP-homologous protein (CHOP) is a key component of the endoplasmic reticulum stress-induced terminal UPR5, 16, 17 and is activated mainly by the PERK pathway, although the IRE1a and ATF6 pathways also contribute.^{5, 18} CHOP deletion has been shown to increase tumorigenesis in mouse models of lung and liver cancers.^{19, 20} Therefore, if the activation of the terminal unfolded protein response triggers cancer cell death, then compounds that enhance the expression or activity of CHOP in cancer cells would induce apoptosis and cell death.

U.S. Pat. No. 4,268,449, entitled, “Method for the preparation of furan-2-carboxylic acid amide and the corresponding furan-2-carboxylic acid,” discloses furan-2-carboxylic acid-amide and the corresponding furan-2-carboxylic acid prepared by contacting carbamoyl chloride and furan at a temperature in the range of from about 10° to about 30° C. in a suitable reaction medium.

### SUMMARY OF THE INVENTION

The present invention provides compositions including small molecule inducers of CHOP expression capable of inducing apoptosis of triple negative breast cancer cells. Using a high-throughput screening assay with HEK293 cells expressing a CHOP promoter-luciferase (CHOP-Luc) reporter, several 5-nitrofuran-2-amide derivatives that induced CHOP-Luc activity were identified.

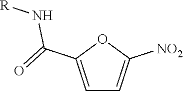

These compounds induced apoptosis in multiple triple negative breast cancer cell lines by inducing CHOP gene expression. Structure-activity relationship (SAR) studies indicated that compounds with an N-phenyl-5-nitrofuran-2-carboxamide skeleton were particularly potent inducers of triple negative breast cancer cell apoptosis.

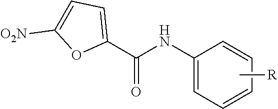

These derivative compounds preferentially activate the eukaryotic initiation factor-2α (eIF2α)-activating transcription factor 4 (ATF4) pathway to induce CHOP expression. The present invention illustrates that augmentation of the terminal unfolded protein response pathway serves as a treatment for cancers with adaptive unfolded protein response activation.

The present invention provides a method of increasing expression of mRNA levels of the endogenous CHOP gene in a cell by providing one or more cells in need of increased mRNA express of a CHOP gene; and administering an effective amount of an N-phenyl-5-nitrofuran-2-carboxamide composition to the one or more cells, wherein the N-phenyl-5-nitrofuran-2-carboxamide composition increases CHOP gene expression. The N-phenyl-5-nitrofuran-2-carboxamide composition activates an eukaryotic initiation factor-2a (eIF2α)-activating transcription factor 4 (ATF4) pathway to induce CHOP expression.

The present invention provides a method of activating a PERK-eIF2α-ATF4 branch of an unfolded protein response expression in a cell by providing one or more cells in need of increasing PERK-eIF2α-ATF4 branch of an unfolded protein response expression; and administering an effective amount of an N-phenyl-5-nitrofuran-2-carboxamide composition to the one or more cells; and increasing PERK-eIF2α-ATF4 branch of an unfolded protein response expression in the one or more cells.

The present invention provides a method of inducing cell apoptosis by providing one or more cells; administering an effective amount of an N-phenyl-5-nitrofuran-2-carboxamide composition to the one or more cells; and increasing the expression of mRNA of a CHOP gene to increase apoptosis in the one or more cells.

The present invention provides a method of treating one or more cancer cells comprising the steps of: providing one or more cancer cells; and administering an effective amount of an N-phenyl-5-nitrofuran-2-carboxamide composition, wherein the N-phenyl-5-nitrofuran-2-carboxamide composition increases the mRNA level of a CHOP gene to increase apoptosis in the one or more cancer cells to treat the one or more cancer cells.

The N-phenyl-5-nitrofuran-2-carboxamide composition has the formula:

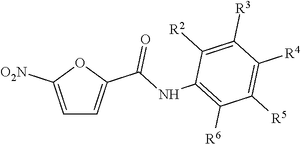

where R4 is a methyl, an ethyl, Cl, Br, I, or F; and R2, R3, R5, and R6 are Hydrogens; R4 is a methyl; and R2, R3, R5, and R6 are Hydrogens; R4 is a ethyl; and R2, R3, R5, and R6 are Hydrogens; R4 is a Cl; and R2, R3, R5, and R6 are Hydrogens; R3 is a meOCF₃ group; and R2, R4, R5, and R6 are Hydrogens; R2 and R5 are Cl, Br, I, or F; and R3, R4, and R6 are Hydrogens; R2 and R5 are methyls or ethyls; R4 is a Br, Cl, I, or F; and R3, and R6 are Hydrogens; R2 is a Cl, Br, I or F; R5 is a CF₃; and R3, R4, and R6 are Hydrogens; R4 is a morpholine; R3 is a hydrogen; and R2, R5, and R6 are hydrogens; R4 is a morpholine; R3 is a Cl, Br, I, or F; and R2, R5, and R6 are hydrogens; R4 is a piperidine; R3 is a Cl, Br, I or F; and R2, R5, and R6 are hydrogens; R4 is a 4 methyl piperidine; R3 is a hydrogen; and R2, R5, and R6 are hydrogens; or R4 is a piperazine; R3 is a hydrogen; and R2, R5, and R6 are hydrogens.

The present invention also includes a therapeutic composition comprising a pharmaceutically acceptable carrier and a therapeutically effective amount of a N-phenyl-5-nitrofuran-2-carboxamide composition has the formula:

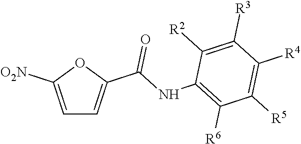

where R4 is a methyl, an ethyl, Cl, Br, I, or F; and R2, R3, R5, and R6 are Hydrogens; R4 is a methyl; and R2, R3, R5, and R6 are Hydrogens; R4 is a ethyl; and R2, R3, R5, and R6 are Hydrogens; R4 is a Cl; and R2, R3, R5, and R6 are Hydrogens; R3 is a meOCF₃ group; and R2, R4, R5, and R6 are Hydrogens; R2 and R5 are Cl, Br, I, or F; and R3, R4, and R6 are Hydrogens; R2 and R5 are methyls or ethyls; R4 is a Br, Cl, I, or F; and R3, and R6 are Hydrogens; R2 is a Cl, Br, I, or F; R5 is a CF₃; and R3, R4, and R6 are Hydrogens; R4 is a morpholine; R3 is a hydrogen; and R2, R5, and R6 are hydrogens; R4 is a morpholine; R3 is a Cl, Br, I or F; and R2, R5, and R6 are hydrogens; R4 is a piperidine; R3 is a Cl, Br, I or F; and R2, R5, and R6 are hydrogens; R4 is a 4 methyl piperidine; R3 is a hydrogen; and R2, R5, and R6 are hydrogens; or R4 is a piperazine; R3 is a hydrogen; and R2, R5, and R6 are hydrogens.

The N-phenyl-5-nitrofuran-2-carboxamide composition may increase the expression level of a CHOP gene mRNA, increase in the CHOP protein expression level in the cell, increased activity of CHOP protein, activate a PERK-eIF2α-ATF4 branch of an unfolded protein response expression in a cell.

In addition, the therapeutic composition may include a targeting molecule that binds to a cell or a portion of a cell and the pharmaceutically acceptable carrier may be a polymer, a liposome, peptide, synthetic composition or a combination thereof.

### BRIEF DESCRIPTION OF THE DRAWINGS

For a more complete understanding of the features and advantages of the present invention, reference is now made to the detailed description of the invention along with the accompanying figures and in which:

FIG. 1A is an image of the structure of one 5-nitrofuran-2-amide derivative.

FIG. 1B is a graph showing luciferase activity of cells treated with DMSO (control) and the compound of FIG. 1A.

FIG. 1C is a graph showing CHOP mRNA expression levels in HEK293 cells treated with DMSO or the compound of FIG. 1A.

FIG. 2A is an image showing apoptosis of HCC-1806 cells after treatment with a 5-nitrofuran-2-amide derivative.

FIG. 2B is a live-cell phase-contrast image showing treatment with DMSO.

FIG. 2C is a live-cell phase-contrast image showing treatment with a 5-nitrofuran-2-amide derivative.

FIG. 3A is a graph showing CHOP mRNA expression levels in HCC-1806 cells treated with a 5-nitrofuran-2-amide derivative.

FIG. 3B is an image showing CHOP mRNA expression levels after treatment with a 5-nitrofuran-2-amide derivative.

FIG. 3C is a graph showing ATF4 mRNA expression levels HEK293 cells treated with DMSO or the compound of a 5-nitrofuran-2-amide derivative.

FIG. 3D is an image showing ATF4 and p-eIF2a protein levels after treatment with a 5-nitrofuran-2-amide derivative.

FIG. 3E is an image showing XBP1 mRNA levels in HCC-1806 cells treated with tunicamycin or a 5-nitrofuran-2-amide derivative.

FIG. 3F is a table showing the quantification of data shown in FIG. 3A by densitometry.

FIG. 3G is a graph showing HEK293 cells stably expressing CHOP-Luc, ERSE-Luc, or UPRE-luc reporters treated with DMSO, a 5-nitrofuran-2-amide derivative, or tunicamycin.

FIG. 3H is a graph showing cell viability of HCC-1806 cells transfected with a control or CHOP siRNA after treatment with a 5-nitrofuran-2-amide derivative or DMSO.

FIG. 4 is a graph of the CHOP expression in HCC-1806 cells in a time-dependent manner induced by a 5-nitrofuran-2-amide derivative.

FIG. 5A is an image showing concentrations of CHOP siRNA or scrambled siRNA transfected into HCC-1806 cells.

FIG. 5B is a table of CHOP knockdown efficiency with the indicated concentrations of CHOP siRNA relative to control siRNA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “Alkyl” refers to a straight or branched hydrocarbon chain group consisting solely of carbon and hydrogen atoms, containing no unsaturation and including, for example, from one to ten carbon atoms, such as 1, 2, 3, 4, 5, 6, 7, 8, 9, or 10 carbon atoms, and which is attached to the rest of the molecule by a single bond. Unless stated otherwise, specifically in the specification, the alkyl group may be optionally substituted by one or more substituents as described herein. Unless stated otherwise specifically herein, it is understood that the substitution can occur on any carbon of the alkyl group.

As used herein the term “Aryl” may be used interchangeably with “aromatic group” or “aromatic ring” and refers to carbocyclic aryl groups, such as phenyl, naphthyl, etc. Unless stated otherwise, specifically herein, the term “aryl” is meant to include aryl groups optionally substituted by one or more substituents as described herein. In some embodiments, the aryl groups may be heteroaryl groups.

As used herein the term “Heteroaryl” refers to a single aromatic ring group containing one or more heteroatoms in the ring, for example N, O, S, including for example, 5-6 members.

As used herein the term “Cycloalkyl” refers to a stable monovalent monocyclic, bicyclic, or tricyclic hydrocarbon group consisting solely of carbon and hydrogen atoms, having for example from 3 to 15 carbon atoms, and which is saturated and attached to the rest of the molecule by a single bond. Unless otherwise stated specifically herein, the term “cycloalkyl” is meant to include cycloalkyl groups which are optionally substituted as described herein.

As used herein the term “Cancer” denotes any unwanted and abnormal growth of any cell type or tissue. In general, a cancer cell has been released from its normal cell division control, i.e., a cell whose growth is not regulated by the ordinary biochemical and physical influences in the cellular environment. In general, a cancer cell proliferates to form a clone of cells which are malignant. The term cancer includes cell growths that are technically benign but which carry the risk of becoming malignant. This term also includes any transformed and immortalized cells cancers, carcinomas, neoplasms, neoplasias, or tumors. In some embodiments, the term cancer refers to solid tumors. Cancers include, for example and without limitation, fibrosarcoma, myosarcoma, liposarcoma, chondrosarcoma, osteogenic sarcoma, chordoma, angiosarcoma, endotheliosarcoma, lymphangiosarcoma, lymphangio and otheliosarcoma, synoviome, mesothelioma, Ewing's tumor, leiomyosarcoma, rhabdomyosarcoma, gastric cancer, esophageal cancer, colon carcinoma, rectal cancer, colorectal cancer, pancreatic cancer, breast cancer, triple negative breast cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinome, medullary carcinoma, bronchogenic carcinoma, renal cell carcinoma, myeloma, hepatoma, hepatocellular cancer, ductal cancer, bile duct carcinoma, choriocarcinoma, seminoma, embryonal carcinoma, Wilm's tumor, liver cancer, cervical cancer, testicular cancer, lung carcinoma, small cell lung carcinoma, non-small cell lung carcinoma, bladder carcinoma, epithelial carcinoma, neural cancer, glioma, astracytoma, medulloblastoma, craniopharyngioma, ependymoma, pinealoma, hemangloblastoma, acoustic neuroma, oligodendroglioma, meningioma, melanoma, neuroblastoma, retinoblastoma, leukemia, chronic myeloid leukemia, lymphoma, Burkitt's lymphoma, or Kaposi's sarcoma.

An “Effective Amount” of a compound according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a compound may vary according to factors such as the disease state, age, sex, and weight of the individual, and the ability of the compound to elicit a desired response in the individual. Dosage regimens may be adjusted to provide the optimum therapeutic response.

As used herein the terms “Optional” or “Optionally” mean that the subsequently described event of circumstances may or may not occur, and that the description includes instances where said event or circumstance occurs one or more times and instances in which it does not. Certain groups may be optionally substituted as described herein. Suitable substituents include: H, alkyl (C 1-6), alkenyl (C 2-6), or alkynyl (C 2-6) each of which may optionally contain one or more heteroatoms selected from O, S, P, N, F, Cl, Br, I, or B. III includes one or more heteroatoms selected from O, S, P, N, F, Cl, Br, I, or B.

As used herein “Pharmaceutically Acceptable Carrier” or “Excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having, or at risk for having, a disorder or condition, be diagnosed with a disorder or condition, or be a control subject that is confirmed to not have a disorder or condition.

The transcription factor C/EBP-homologous protein (CHOP) is a key component of the terminal unfolded protein response (UPR) that mediates unresolvable endoplasmic reticulum stress-induced apoptosis. CHOP induction is known to cause cancer cell death. Chemicals that induce CHOP expression are valuable as potential cancer therapeutics and as research tools. The present inventors discovered that 5-nitrofuran-2-amide derivatives function as small molecule activators of CHOP expression that induced apoptosis in triple negative breast cancer (TNBC) cells. Structure-activity relationship studies indicated that compounds with an N-phenyl-5-nitrofuran-2-carboxamide skeleton were particularly potent inducers of TNBC cell apoptosis. The compounds activate CHOP expression via the PERK-eIF2α-ATF4 branch of the unfolded protein response. These results indicate that small molecule activators of CHOP expression have therapeutic potential for TNBC.

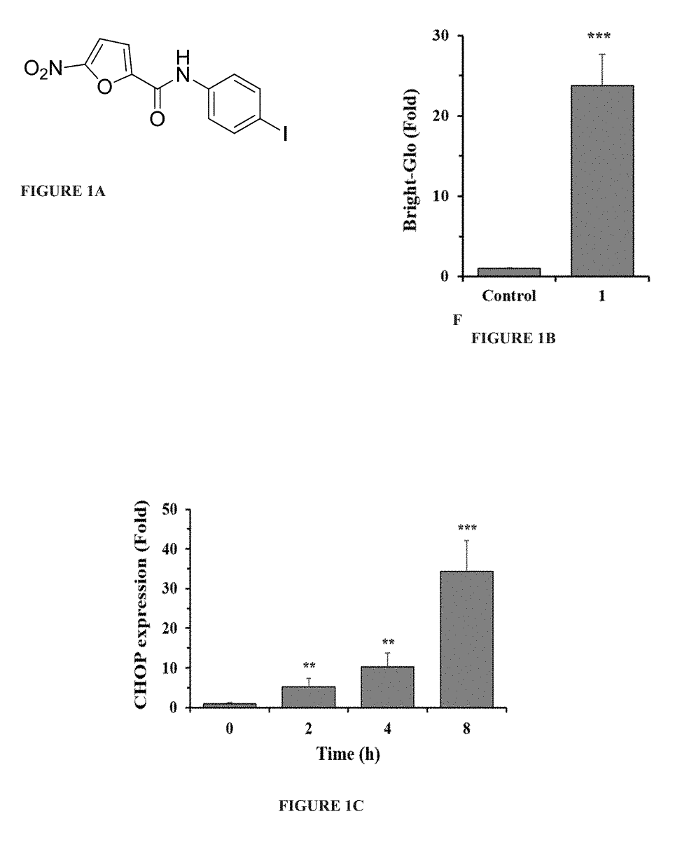

FIG. 1A is an image of the structure of a N-phenyl-5-nitrofuran-2-carboxamide derivative and more specifically compound 1 or N-(4-iodophenyl)-5-nitrofuran-2-carboxamide:

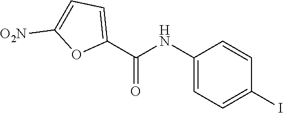 [SMILES: O=C(Nc1ccc(I)cc1)c1ccc([N+](=O)[O-])o1]

FIG. 1B is a graph showing luciferase activity of cells treated with DMSO (control) and N-(4-iodophenyl)-5-nitrofuran-2-carboxamide (10 μM) for 24 h, and luciferase activity was determined using the Bright-Glo assay.

FIG. 1C is a graph showing CHOP mRNA expression levels in HEK293 cells were treated with DMSO or N-(4-iodophenyl)-5-nitrofuran-2-carboxamide (10 μM) for 24 h and CHOP mRNA levels were analyzed by qRT-PCR. The results are the means of 3 replicate wells and are representative of 3 independent experiments. **P<0.01 and ***P<0.001 by Student's t-test compared with control cells.

To identify compounds that activate the expression of CHOP, a HEK293 cell line stably expressing a CHOP-Luc reporter construct that faithfully reflects endogenous CHOP gene expression²¹ was used to screen approximately 50,000 structurally diverse small molecules. In one embodiment a novel compound 1, N-(4-iodophenyl)-5-nitrofuran-2-carboxamide was identified that increased the activity of the CHOP-Luc reporter by 24-fold at the concentration of 10 μM (FIGS. 1A, 1B). The inventor further determined whether N-(4-iodophenyl)-5-nitrofuran-2-carboxamide affects the expression of the endogenous CHOP gene. As shown in FIG. 1C, N-(4-iodophenyl)-5-nitrofuran-2-carboxamide significantly increased the expression of mRNA level of the endogenous CHOP gene in HEK293 cells, by up to 30-fold increase, as measured by quantitative RT-PCR (FIG. 1C).

Given the known functions of CHOP in ER stress-induced apoptosis^{16, 17} and in regulating cancer cell death,^{6, 19, 20} the inventor investigated whether compound 1 affects the viability of TNBC cells. Three human TNBC cell lines, HCC-1806, HCC-1143, and HCC-38, were treated with doses of 1 ranging from 0.125 μM to 20 μM and their viability was assessed using the CellTiter-Glo assay, which measures intracellular ATP levels. The viability of the three TNBC cell lines was significantly reduced by 1 in a dose-dependent manner, and the IC50 values were similar on all three cell lines; 6.2 μM, 9.5 μM, and 8.7 μM, for HCC-1806, HCC-1143, and HCC-38 cells, respectively. These results indicate that N-(4-iodophenyl)-5-nitrofuran-2-carboxamide exhibits antitumor activity in TNBC cells.

Other embodiments of 5-nitrofuran-2-amide derivatives were identified by performing SAR analysis on a series of N-(4-iodophenyl)-5-nitrofuran-2-carboxamide analogues and assessed their antitumor activity using the HCC-1806 cell line. The effects on efficacy of various substituted groups introduced to the phenyl ring were evaluated. Anticancer effect of 2a-2l in HCC-1806 cells as shown in Table 1 below.

[symbol]

^{a} IC₅₀ value for cancer cell viability calculated with GraphPad Prism. Replacement of the 4-iodine group with methyl, ethyl, or chlorine appeared to have no effect on or slightly improved the antitumor activity, as indicated by the similar IC50s of the respective compounds 2a, 2b, and 2c (see Table 1 above) compared to that of N-(4-iodophenyl)-5-nitrofuran-2-carboxamide. In contrast, 2,6-di-Me and 2-iBu replacement resulted in inactive analogues 2k and 2l, suggesting that steric hindrance reduces the potency. In addition, introduction of 2,5-dimethyl (2f) or 2-ethyl (2i) to the phenyl ring in 1 moderately improved the potency.

Given that the substituents at the para position of the phenyl ring were well tolerated, various six-ring substituents were introduced to the phenyl ring, and the compounds were tested for their anti-TNBC activity in HCC-1806, HCC-1143 and HCC-38 cells are shown in Table 2 below.

[symbol]

TABLE 2

| | | | HCC-1806 | HCC-1143 | HCC-38 |
|---|---|---|---|---|---|
| Compound | R₁ | R₂ | IC₅₀ (μM) ^{a} | IC₅₀ (μM) ^{a} | IC₅₀ (μM) ^{a} |
| 3a | 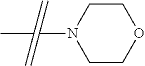 | H | 1.5 | 2.3 | 3.1 |
| 3b | 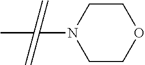 | Cl | 2.6 | 2.5 | 2.0 |
| 3c | 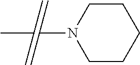 | Cl | 3.5 | 4.5 | 3.8 |
| 3d | 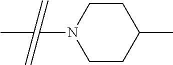 | H | 2.8 | 2.1 | 2.5 |
| 3e | 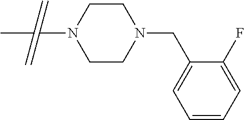 | H | 3.0 | 3.8 | 1.9 |

^{a} IC₅₀ value for cancer cell viability calculated with GraphPad Prism. All of the six-ring derivatives tested, including morpholine (3a, 3b), piperidine (3c, 3d), and piperazine (3e), exhibited substantially improved IC50 values for inhibition of HCC-1806 viability. Compounds 3a-e all also showed significant activity on HCC-1143 and HCC-38.

Further SAR analysis indicated that the nitro group on the left furan ring is critical for the anticancer activity, since its deletion in compounds 4a-4d eliminated or severely inhibited their activity in HCC-1806 cells as shown in Table 3 below.

[symbol]

TABLE 3

| | | | HCC-1806 |
|---|---|---|---|
| Compound | R₁ | R₂ | IC₅₀ (μM) ^{a} |
| 4a | 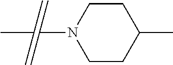 | H | >40 |
| 4b | 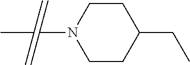 | Cl | >40 |
| 4c | 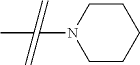 | F | >40 |
| 4d | 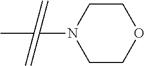 | Br | >40 |

^{a} IC₅₀ value for cancer cell viability calculated with GraphPad Prism. SAR studies indicated that compounds with an N-phenyl-5-nitrofuran-2-carboxamide skeleton were potent inhibitors of TNBC cell viability.

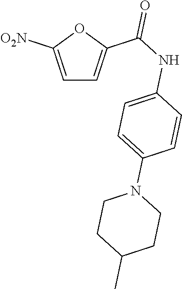 [SMILES: CC1CCN(c2ccc(NC(=O)c3ccc([N+](=O)[O-])o3)cc2)CC1]

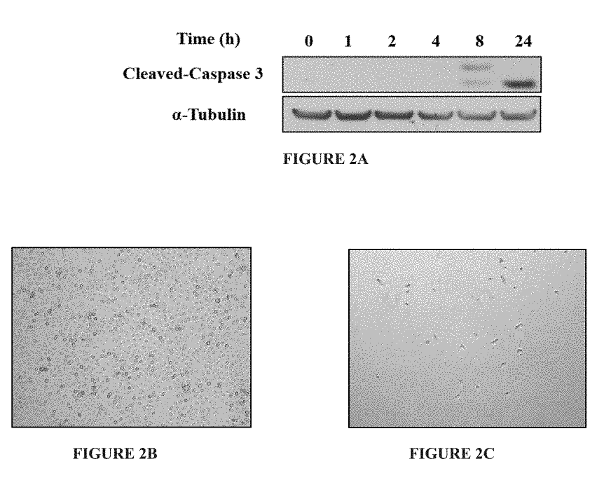

FIG. 2A is an image showing apoptosis of HCC-1806 cells after treatment with a 5-nitrofuran-2-amide derivative compound 3d (above). Cells were treated with 3d (10 μM) for the indicated times, and cleavage of caspase-3 was determined by Western blotting. α-Tubulin was used as a loading control. The data shown are representative of 3 independent studies. FIGS. 2B and 2C are live-cell phase-contrast images (magnification 10×) showing treatment with DMSO and (10 μM) 5-nitrofuran-2-amide derivative compound 3d respectively for 24 h. To determine whether the reduction in TNBC cell viability by the 5-nitrofuran-2-amide derivatives was due to the induction of apoptosis, the inventor analyzed cleavage of caspase-3, a critical executioner of apoptosis, in HCC-1806 cells treated with compound 3d. Indeed, treatment of HCC-1806 cells with compound 3d increased cleavage of caspase-3 protein levels at 8 h and 24 h, indicating that 3d activated apoptosis in the TNBC cells (see FIG. 2A). To confirm this, HCC-1806 cells were cultured to near confluence and treated with 10 μM 3d or DMSO for 24 h and then imaged by live-cell phase-contrast microscopy. Whereas the DMSO-treated cells remained confluent, few cells were observed in the 3d-treated culture, indicative of significant cell death and thus, detachment from the culture dish, rather than a reduction in proliferation, (as shown in FIGS. 2B and 2C).

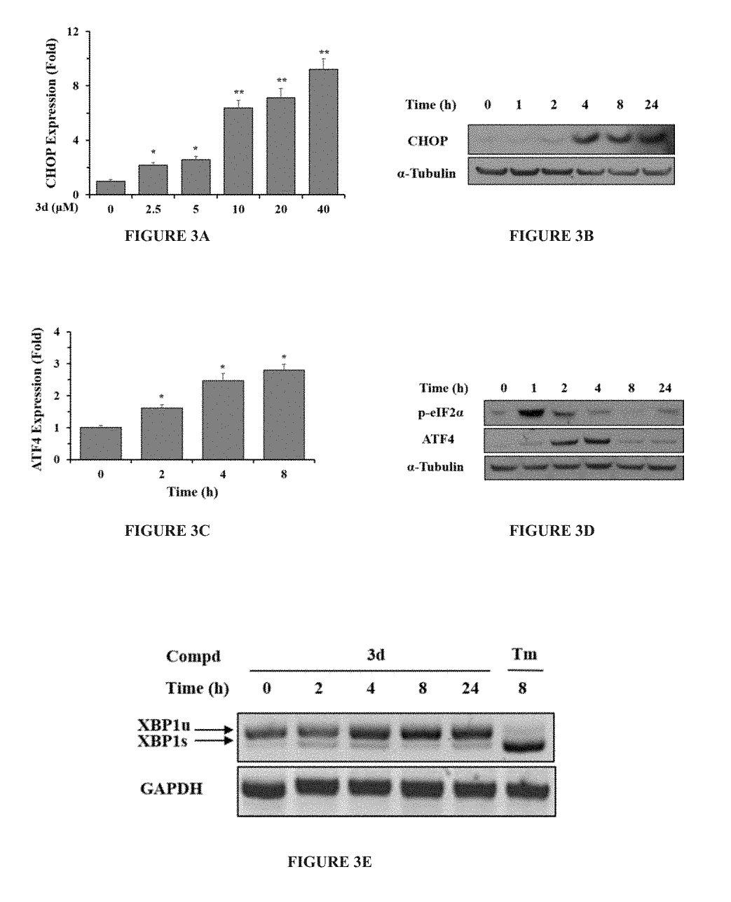

FIG. 3A is a graph showing CHOP mRNA expression levels in HCC-1806 cells by selectively activating eIF2α-ATF4 pathway when treated with a 5-nitrofuran-2-amide derivative compound 3d. Cells were treated with compound 3d at the indicated concentrations for 8 h, and CHOP mRNA levels were analyzed by qRT-PCR. The results are the means of 4 replicate wells and are representative of 3 independent studies. *P<0.05 and **P<0.01 by Student's t-test compared with cells treated with DMSO (in FIG. 3A) or with 3d for 0 h (in FIG. 3B).

FIG. 3B is an image showing CHOP mRNA expression levels after treatment with a 5-nitrofuran-2-amide derivative. Cells were treated with compound 3d (10 μM) for the indicated times, and CHOP protein levels were analyzed by Western blotting. α-Tubulin was used as a loading control. The data shown are representative of 3 independent studies.

FIG. 3C is a graph showing ATF4 mRNA expression levels HEK293 cells were treated with DMSO or compound 3d. Cells were treated with compound 3d (10 μM) for the indicated times, and ATF4 mRNA levels were analyzed by qRT-PCR. Results are the means of 4 replicate wells and are representative of 3 independent studies. *P<0.05 by Student's t-test compared with DMSO-treated cells.

FIG. 3D is an image showing ATF4 and p-EIF2α protein levels after treatment with a 5-nitrofuran-2-amide derivative compound 3d. Cells were treated with compound 3d (10 μM) for the indicated times, and ATF4 and p-eIF2a protein levels were analyzed by Western blotting. α-Tubulin was used as a loading control. The data shown are representative of 3 independent studies.

FIG. 3E is a graph showing XBP1 mRNA levels in HCC-1806 cells treated with tunicamycin or a 5-nitrofuran-2-amide derivative compound 3d. HCC-1806 cells were treated with compound 3d (10 μM) or tunicamycin (Tm, 1 μg/mL) for the indicated times. XBP1 mRNA levels were analyzed by RT-PCR and the products were resolved by agarose gel electrophoresis. The full-length (unspliced, XBP1u) and spliced (XBP1s) forms of XBP1 mRNA are indicated. GAPDH mRNA was used as an internal control.

FIG. 3F is a table showing the quantification of data shown in FIG. 3A by densitometry. The percentage of XBP1s relative to total XBP1 was calculated as: (XBP1s/[XBP1s+XBP1u])×100%. The data shown are representative of 3 independent studies.

FIG. 3G is a graph showing HEK293 cells stably expressing CHOP-Luc, ERSE-Luc, or UPRE-luc reporters were treated with DMSO, a 5-nitrofuran-2-amide derivative, or tunicamycin. HEK293 cells stably expressing CHOP-Luc, ERSE-Luc, or UPRE-luc reporters were treated with DMSO, compound 3d (10 μM), or Tm (1 μg/mL) for 24 h, and luciferase activity was measured using the Bright-Glo assay. Results are the means of 4 replicate wells and are representative of 3 independent experiments.

FIG. 3H is a graph showing cell viability of HCC-1806 cells transfected with a control or CHOP siRNA after treatment with a 5-nitrofuran-2-amide derivative or DMSO. Control or CHOP siRNA (20 nM) was transfected into HCC-1806 cells, and 6 h later, cells were treated with compound 3d (5 μM) or DMSO for 48 h. Cell viability was measured using the CellTiter-Glo assay. The data shown are representative of 3 independent studies. *P<0.05 by Student's t-test compared with DMSO-treated cells.

The effect of compound 3d on CHOP gene expression in HCC-1806 cells was examined using quantitative real-time PCR. Treatment of cells with compound 3d significantly increased CHOP mRNA levels in a dose-dependent manner (see FIGS. 3A-3H).

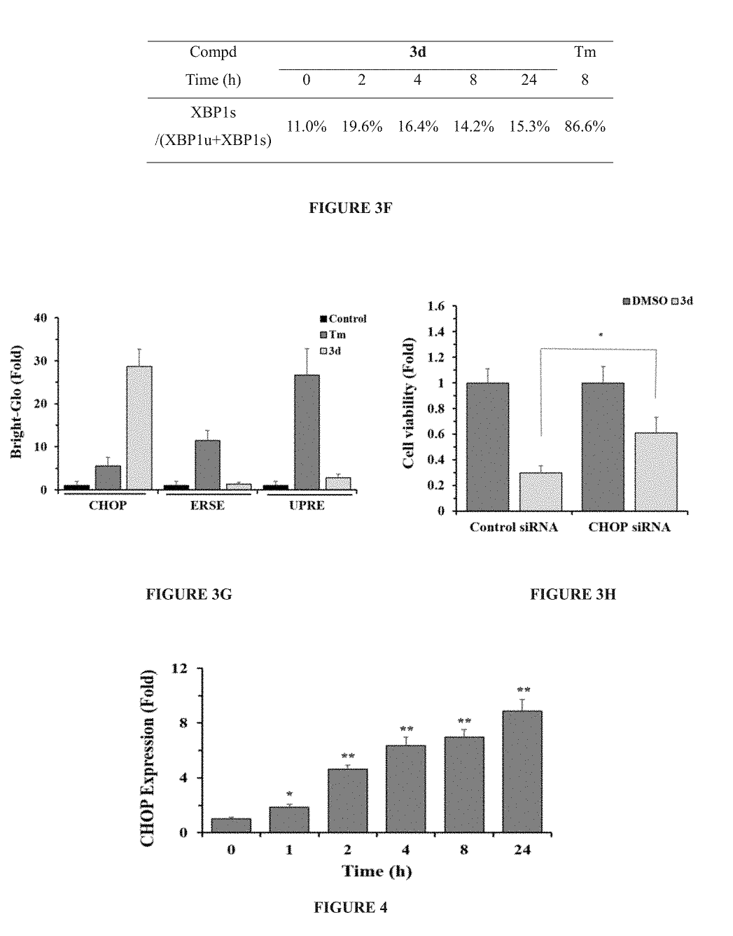

FIG. 4 is a graph of the CHOP expression in HCC-1806 cells in a time-dependent manner induced by compound 3d. Cells were treated with compound 3d (10 μM) for the indicated times, and CHOP mRNA levels were analyzed by qRT-PCR. The results are the means of 4 replicate wells and are representative of 3 independent studies. *P<0.05 and **P<0.01 by Student's t-test compared with cells treated with compound 3d for 0 h.

Similarly, a kinetic analysis revealed that treatment with 10 μM compound 3d increased CHOP transcription in a time-dependent fashion. FIG. 4 is a graph of the CHOP expression in HCC-1806 cells in a time-dependent manner induced by a 5-nitrofuran-2-amide derivative. Cells were treated with compound 3d (10 μM) for the indicated times, and CHOP mRNA levels were analyzed by qRT-PCR. The results are the means of 4 replicate wells and are representative of 3 independent studies. *P<0.05 and **P<0.01 by Student's t-test compared with cells treated with compound 3d for 0 h.

In both dose- and time-dependent studies, compound 3d induced CHOP mRNA levels were up to 9-fold higher than in DMSO-treated cells. In agreement with its effects on CHOP transcription, compound 3d treatment of HCC-1086 cells also increased CHOP protein levels, with significant increases detected between 4 h and 24 h (See FIG. 3B). These results demonstrate that compound 3d activates the expression of the CHOP gene in TNBC cells.

Prolonged or severe ER stress activates CHOP expression primarily through the PERK branch of the unfolded protein response, although the IRE1α and ATF6 branches also contribute.^{5, 18} The 5-nitrofuran-2-amide derivatives could induce CHOP expression by acting as an ER stressor that activates all three branches of unfolded protein response or by preferentially activating a select branch of unfolded protein response. To distinguish between these possibilities, the inventor investigated which branches of the unfolded protein response were affected by compound 3d. Activation of the PERK pathway by ER stress leads to phosphorylation of eIF2α, followed by activation of the transcription and translation of the transcription factor ATF4, a 5′-upstream ORF-containing gene, and ATF4-mediated CHOP expression.^{5, 22} To determine whether the PERK pathway is involved in compound 3d-mediated CHOP induction, ATF4 expression and eIF2a phosphorylation in HCC-1806 cells by qRT-PCR and Western blotting were analyzed. Compound 3d significantly increased ATF4 expression at both the mRNA (See FIG. 3C) and protein (See FIG. 3D) levels, and substantially increased the phosphorylation of eIF2a (See FIG. 3D). Of note, the compound 3d-induced increase in eIF2a phosphorylation preceded the increase in ATF4 mRNA, and both effects peaked within several hours of compound 3d treatment; a pattern consistent with a typical ER stress-mediated response.⁶ These results support a role for the PERK-eIF2α-ATF4-CHOP branch of the unfolded protein response in compound 3d-mediated TNBC cancer cell death.

The inventor next asked whether the IRE1α and ATF6 branches of the unfolded protein response were also activated by compound 3d treatment of TNBC cells. Under ER stress, activated IRE1α cleaves X-box binding protein-1 (XBP1) mRNA to generate a spliced form of XBP1 that is translated into a potent transcription factor XBP1s (for spliced XBP1).¹⁴ XBP1s increases transcription of unfolded protein response genes encoding factors involved in ER protein folding and degradation by binding to the unfolded protein response element (UPRE) in the gene promoters, either as a homodimer or as an XBP1s-ATF6 heterodimer.^{23, 24} XBP1 mRNA splicing and a UPRE-Luciferase (UPRE-Luc) reporter as markers for activation of IRE1α pathway were used. XBP1 mRNA splicing, as measured by XBP1s levels, increased only slightly in the first 24 h after compound 3d treatment of HCC-1806 cells (from 11% of total XBP1 at 0 h to 15˜20% at 2˜24 h). This compared with the dramatic increase of XBP1s (˜87% at 8 h) after treatment with tunicamycin, a well-characterized ER stressor (See FIG. 3E, 3F). The effect of compound 3d treatment on the IRE1α pathway was analyzed using a HEK293 UPRE-Luc reporter cell line, and it was found that luciferase activity was increased less than 2-fold by compound 3d, compared with >25-fold by tunicamycin (See FIG. 3G). These results suggest that activation of the IRE1α pathway is likely to contribute marginally, if at all, to the anticancer activity of compound 3d. Next, it was determined whether compound 3d activates the ATF6 pathway by evaluating its effect on the activity of an ER stress response elements (ERSE)-Luc reporter stably established in HEK293 cell line. Under ER stress, activated ATF6 functions as a nuclear transcription factor and activates the expression of genes encoding ER chaperones by binding to ERSEs in their promoters.^{24, 25} Whereas ERSE-driven luciferase activity was increased by ˜12-fold upon treatment with tunicamycin, treatment with compound 3d for 24 h had no significant effect (FIG. 3F). Taken together, these results indicate that compound 3d does not behave as a general ER stressor to activate all 3 branches of the unfolded protein response (e.g., tunicamycin), but instead, induces CHOP expression by selectively activating the PERK-eIF2α-ATF4 branch of the unfolded protein response.

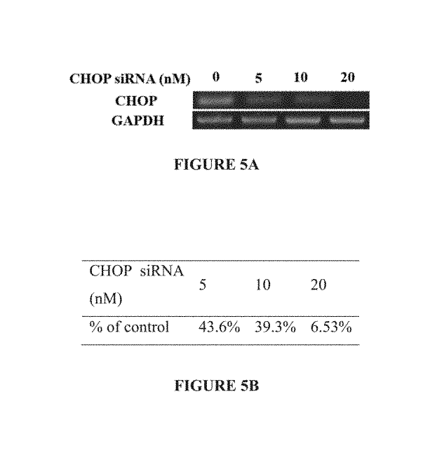

FIG. 5A is an image showing concentrations of CHOP siRNA or scrambled siRNA were transfected into HCC-1806 cells. The indicated concentrations of CHOP siRNA or scrambled siRNA (indicated as 0 nM) were transfected into HCC-1806 cells for 48 h, and CHOP mRNA levels were analyzed by RT-PCR. GAPDH mRNA was used as an internal control. FIG. 5B is a table of CHOP knockdown efficiency with the indicated concentrations of CHOP siRNA relative to control siRNA.

To confirm that CHOP induction plays a role in compound 3d-mediated TNBC cell death, the inventor asked whether siRNA-mediated knockdown of CHOP mitigated compound 3d-induced cell death. For this, HCC-1806 cells were transfected with different concentrations of CHOP siRNA, and knockdown efficiency was assessed by RT-PCR. CHOP mRNA levels were reduced by >90% at 48 h after transfection with 20 nM CHOP-specific siRNA compared with control siRNA (See FIGS. 5A and 5B), and neither siRNA had a significant effect on cell viability under control conditions (See FIG. 3H). As expected, compound 3d caused a marked reduction in the viability of cells transfected with the control siRNA; however, CHOP siRNA significantly attenuated the effects of compound 3d on HCC-1806 cell death (See FIG. 3H). These results indicate that CHOP is critical for compound 3d-induced TNBC cell death.

One embodiment of the present invention provides numerous 5-nitrofuran-2-amide derivatives that induced expression of CHOP, a key component of the pro-apoptotic arm of the unfolded protein response. The derivatives induce CHOP expression by preferentially activating the PERK-eIF2α-ATF4 branch of the unfolded protein response; an observation that suggests a highly selective mode of action. 5-nitrofuran-2-amide derivatives were previously reported to identification of similar small molecules as novel activators of CHOP expression leads to the development of new classes of therapeutics for drug-resistant TNBCs.

The chemical libraries were obtained from ChemBridge (San Diego, Calif., US), Maybridge (Cornwall, UK), and MicroSource (Ann Arbor, Mich., US). The compounds were supplied as 10 mM solutions in DMSO. All 5-nitrofuran-2-amide derivatives were obtained from ChemBridge. The structures and purities were confirmed by the suppliers using NMR and HPLC. Tunicamycin was obtained from Sigma (St Louis, Mo., US). All chemicals were dissolved in DMSO and used at the indicated concentrations. Bright-Glo and CellTiter-Glo kits were purchased from Promega (Madison, Wis., US).

HEK293T cells were cultured in DMEM medium (Corning, N.Y., US) supplemented with 10% fetal bovine serum (FBS; Atlanta Biologicals, Norcross, Ga.), and antibiotics (100 UI/mL penicillin and 100 μg/mL streptomycin; Corning) and maintained in a humidified 5% CO₂ atmosphere at 37° C. HCC-1806, HCC-1143, and HCC-38 cells (from ATCC) were cultured in RPMI 1640 medium (Corning) with 10% FBS (Atlanta Biologicals) and antibiotics (100 UI/mL penicillin and 100 μg/mL streptomycin; Corning) and maintained in a humidified 5% CO₂ atmosphere at 37° C.

The HEK293T CHOP reporter cell line (CHOP-Luc) was previously described.²¹ HEK293T cells were stably transfected with ERSE-Luciferase²⁵ and UPRE-Luciferase²³ reporters to generate ERSE-Luc and UPRE-Luc reporter cell lines, respectively. Reporter cells were plated at 7×10³ cells/well in a 384-well plate and incubated for 16 h. Test compounds or Tm at 1 μg/mL were then added. Luciferase activity was measured with a Bright-Glo kit 24 h later.

HEK293T CHOP-Luc cells were seeded at 7×10³ cells/well in 384-well plates and treated with 10 μM of the library compounds the next day. After 24 h treatment, the medium was aspirated and 20 μL/well of Bright-Glo luciferase assay reagent was added. Luminescence was measured with an EnVision multilabel plate reader (PerkinElmer, Waltham, Mass., US). Hit selection was based on standard scores. The mean and standard deviation (SD) of luminescence for each compound was determined, and the standard score for each compound was then calculated as (raw measurement of a compound−mean)/SD of the plate. Compounds that increased ATP levels >3 SD compared with control wells (standard score >3) were considered hits.

HCC-1806, HCC-1143, or HCC-38 cells were seeded at 3×10³ cells/well in a 384-well plate and treated with compounds at the indicated concentrations. After 3 d treatment, the medium was aspirated and 20 μL/well of CellTiter-Glo reagent was added. Cell viability was measured with an EnVision multilabel plate reader. The IC₅₀ value for cell viability of each compound was calculated with GraphPad Prism (La Jolla, Calif., US).

HCC-1806 cells were seeded at 4×10⁵ cells/well in 6-well plates and treated with compounds for the indicated times. Total RNA was extracted using TRIzol reagent (Invitrogen, Carlsbad, Calif.) according to the manufacturer's protocol, and 2 μg of total RNA was reverse transcribed using a Superscript kit (Invitrogen). Real-time PCR was performed in 96-well format using SYBR Select Master Mix (Applied Biosystems, Foster City, Calif.) with an ABI 7500 PCR system (Applied Biosystems).

The primer sequences used were: Human CHOP: SEQ ID NO: 1 F, 5′-GCCTTTCTCTTCG-3′ and SEQ ID NO: 2 R, 5′-TGTGACCTCTGCTGGTTCTG-3′. Human ATF4: SEQ ID NO: 3 F, 5′-TTCTCCAGCGACAAGGCTAAGG-3′ and SEQ ID NO: 4 R, 5′-CTCCAACATCCAATCTGTCCCG-3′. Human XBP1: SEQ ID NO: 5 F, 5′-GCTTGTGATTGAGAACCAGG-3′ and SEQ ID NO: 6 R, 5′-GAAAGG GAGGCTGGTAAGGAAC-3′. Human Cyclophilin A: SEQ ID NO: 7 F, 5′-GCCTCTCCCTAGCTTTGGTT-3′ and SEQ ID NO: 8 R, 5′-GGTCTGTTAAGGTGGGCAGA-3′. Human GAPDH: SEQ ID NO: 9 F, 5′-CACAGTCCATGCCATCACTG-3′ and SEQ ID NO: 10 R, 5′-TACTCCTTGGAGGCCATGTG-3′.

HCC-1806 cells were seeded in 60-mm dishes at 8×10⁵ cells/dish and treated for the indicated times. Cells were then washed with PBS and lysed with lysis buffer (Cell Signaling Technology, Danvers, Mass., US) containing EDTA and phosphatase inhibitors. Aliquots of 20 μg total protein were separated on 7% SDS-PAGE gels (Life Technologies, Carlsbad, Calif., US) and transferred to PVDF membranes. The membranes were probed with primary antibodies followed by the appropriate HRP-conjugated secondary antibodies (goat anti-rabbit IgG and goat anti-mouse IgG, 1:3000; Santa Cruz Biotechnology, Santa Cruz, Calif., US). Blots were then developed. The primary antibodies and dilutions used were: CHOP (1:1000 no. MA1-250; Thermo, IL, US), cleaved caspase 3 (1:1000 no. 9661; Cell Signaling Technology), ATF4 (1:1000 no. 10835-1-AP; ProteinTech Group, IL, US), p-eIF2α (Ser51) (1:1000 no. 9721; Cell Signaling Technology), and α-tubulin (1:2000 no. SC-8035; Santa Cruz Biotechnology).

HCC-1806 cells incubated with serum-free RPMI 1640 medium (Corning) were transfected with scrambled control or CHOP siRNA (E-004819-00; Dharmacon/Thermo Scientific, IL, US) using LipofectAMINE reagent (Invitrogen). After 6 h, the medium was replaced with RPMI 1640 medium supplemented with 10% FBS and compound 3d was added. After 48 h, the medium was aspirated and 60 μL/well of CellTiter-Glo reagent was added in 96-w format. Cell viability was measured with an EnVision multilabel plate reader.

Other embodiments of the present invention may include a compound general chemical formula:

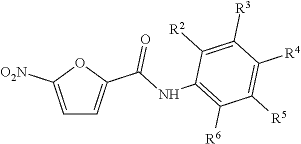

where R1-R6 may be a hydrogen, a halogen, an alkyl, an Aryl or a cycloalkyl having 5-6 carbons or optionally substituted with one or more hetero atoms, e.g., a morpholine, piperidine, 4 methyl piperidine, piperazine.

Examples of heteroaryl groups include furan, thiophene, pyrrole, oxazole, thiazole, imidazole, pyrazole, isoxazole, isothiazole, 1,2,3-oxadiazole, 1,2,3-triazole, 1,2,4-triazole, 1,3,4-thiadiazole, tetrazole, pyridine, pyridazine, pyrimidine, pyrazine, 1,3,5-triazine, imidazole. Unless stated otherwise specifically herein, the term “heteroaryl” is meant to include heteroaryl groups optionally substituted by one or more substituents as described herein. In some embodiments, the aromatic group may be pyridine, thiophene, or benzene.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

Throughout this application, it is contemplated that the term “compound” or “compounds” refers to the compounds discussed herein and includes precursors and derivatives of the compounds, including acyl-protected derivatives, and pharmaceutically acceptable salts of the compounds, precursors, and derivatives. In some embodiments, the invention also includes prodrugs of the compounds, pharmaceutical compositions including the compounds and a pharmaceutically acceptable carrier, and/or pharmaceutical compositions including prodrugs of the compounds and a pharmaceutically acceptable carrier.

In general, compounds described herein may be prepared by standard techniques known in the art, or by known processes analogous thereto. In some embodiments, many of the compounds may be obtained from commercial sources, such as Maybridge, Cornwall, UK

The present disclosure provides methods of treating a disorder or condition resulting in cells with supernumerary centrosomes, such as cancer. The term “treating” as used herein includes treatment, prevention, and amelioration.

In general, the methods are effected by administering a compound as described herein to a subject in need thereof, or by contacting a cell or a sample with a compound as described herein, for example, a pharmaceutical composition comprising a therapeutically effective amount of the compound disclosed herein. More particularly, the compounds are useful in the treatment of a disorder or condition resulting in cells, such as cancer.

In addition the present invention includes a targeting moiety that can direct the composition to a specific cell. The targeting can be accomplished in various methods known to the skilled artisan, for example, U.S. Pat. No. 8,246,968 the contents of which is incorporated herein by reference. By having targeting moieties, the “target specific” nanoparticles are able to efficiently bind to or otherwise associate with a biological entity, for example, a membrane component or cell surface receptor. Targeting (to a particular tissue or cell type, to a specific diseased tissue but not to normal tissue, etc.) of a therapeutic agent of one or more of the compositions disclosed herein is desirable for the treatment of tissue specific diseases such as cancer (e.g. breast cancer). The targeted delivery allows for the administration of a lower dose of the agent, which reduces the undesirable side effects commonly associated with traditional chemotherapy. The target specificity of the composition of the invention can be maximized by optimizing the targeting moiety density.

One targeting moiety may be a nanoparticle, wherein the nanoparticle has a ratio of ligand-bound polymer to non-functionalized polymer effective for the treatment of cancer. For example the composition includes providing a therapeutic agent; providing a polymer; providing a low-molecular weight PSMA ligand; mixing the polymer with the therapeutic agent to prepare particles; and associating the particles with the low-molecular weight PSMA ligand, e.g., a polymer comprises a copolymer of two or more polymers. In another embodiment, the copolymer is a copolymer of PLGA and PEG or PLA and PEG. In other embodiments of the target-specific nanoparticles may include a polymeric matrix of two or more polymers, e.g., polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In still another embodiment, the polymeric matrix comprises one or more polyesters, polyanhydrides, polyethers, polyurethanes, polymethacrylates, polyacrylates or polycyanoacrylates. In another embodiment, at least one polymer is a polyalkylene glycol. In still another embodiment, the polyalkylene glycol is polyethylene glycol. In yet another embodiment, at least one polymer is a polyester. In another embodiment, the polyester is selected from the group consisting of PLGA, PLA, PGA, and polycaprolactones. In still another embodiment, the polyester is PLGA or PLA. In yet another embodiment, the polymeric matrix comprises a copolymer of two or more polymers. In another embodiment, the copolymer is a copolymer of a polyalkylene glycol and a polyester. In still another embodiment, the copolymer is a copolymer of PLGA or PLA and PEG. In yet another embodiment, the polymeric matrix comprises PLGA or PLA and a copolymer of PLGA or PLA and PEG.

Another example of a delivery mechanism that includes a targeting moiety that can direct the composition to a specific cell is a liposome. As used herein, the term “liposome” refers to a generally spherical vesicle or capsid generally comprised of amphipathic molecules (e.g., having both a hydrophobic (nonpolar) portion and a hydrophilic (polar) portion). Typically, the liposome can be produced as a single (unilamellar) closed bilayer or a multicompartment (multilamellar) closed bilayer. The liposome can be formed by natural lipids, synthetic lipids, or a combination thereof. In a preferred embodiment, the liposome comprises one or more phospholipids. Lipids known in the art for forming liposomes include, but are not limited to, lecithin (soy or egg; phosphatidylcholine), dipalmitoylphosphatidylcholine, dimyristoylphosphatidylcholine, distearoylphosphatidylcholine, dicetylphosphate, phosphatidylglycerol, hydrogenated phosphatidylcholine, phosphatidic acid, cholesterol, phosphatidylinositol, a glycolipid, phosphatidylethanolamine, phosphatidylserine, a maleimidyl-derivatized phospholipid (e.g., N-[4(p-malei-midophenyl)butyryl]phosphatidylethanolamine), dioleylphosphatidylcholine, dipalmitoylphosphatidylglycerol, dimyristoylphosphatidic acid, and a combination thereof. Liposomes have been used to deliver therapeutic agents to cells.

Another example of a delivery mechanism that includes a targeting moiety that can direct the composition to a specific cell is dendritic polymers are uniform polymers, variously referred to in the literature as hyperbranched dendrimers, arborols, fractal polymers and starburst dendrimers, having a central core, an interior dendritic (hyperbranched) structure and an exterior surface with end groups.

Another example of a delivery mechanism that includes a targeting moiety that can direct the composition to a specific cell include Albumin particles, siRNA, DNA, proteins, protein mimics, synthetic proteins or portions of proteins.

Compounds and compositions as described herein can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to mammals, for example, humans, cattle, sheep, etc. If desired, treatment with a compound according to the invention may be combined with more traditional and existing therapies for disorders or conditions, such as cancer. Accordingly, in some embodiments, compounds as described herein may be provided in combination with for example mitotic inhibitors, such as paclitaxel, docotaxel, vinblastine, vincristine, vinorelbine, etc. In some embodiments, compounds as described herein may be provided in combination with chemotherapy or radiation therapy.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] Hetz, C. (2012) The unfolded protein response: controlling cell fate decisions under ER stress and beyond, Nature reviews. Molecular cell biology 13, 89-102.
[2] Walter, P., and Ron, D. (2011) The unfolded protein response: from stress pathway to homeostatic regulation, Science 334, 1081-1086.
[3] Wang, S., and Kaufman, R. J. (2012) The impact of the unfolded protein response on human disease, J. Cell Biol. 197, 857-867.
[4] Shore, G. C., Papa, F. R., and Oakes, S. A. (2011) Signaling cell death from the endoplasmic reticulum stress response, Curr. Opin. Cell Biol. 23, 143-149.
[5] Tabas, I., and Ron, D. (2011) Integrating the mechanisms of apoptosis induced by endoplasmic reticulum stress, Nat. Cell Biol. 13, 184-190.
[6] Wang, M., and Kaufman, R. J. (2014) The impact of the endoplasmic reticulum protein-folding environment on cancer development, Nat. Rev. Cancer 14, 581-597.
[7] Verfaillie, T., Salazar, M., Velasco, G., and Agostinis, P. (2010) Linking ER Stress to Autophagy: Potential Implications for Cancer Therapy, International journal of cell biology 2010, 930509.
[8] Chen, X., Iliopoulos, D., Zhang, Q., Tang, Q., Greenblatt, M. B., Hatziapostolou, M., Lim, E., Tam, W. L., Ni, M., Chen, Y., Mai, J., Shen, H., Hu, D. Z., Adoro, S., Hu, B., Song, M., Tan, C., Landis, M. D., Ferrari, M., Shin, S. J., Brown, M., Chang, J. C., Liu, X. S., and Glimcher, L. H. (2014) XBP1 promotes triple-negative breast cancer by controlling the HIF1alpha pathway, Nature 508, 103-107.
[9] Davenport, E. L., Moore, H. E., Dunlop, A. S., Sharp, S. Y., Workman, P., Morgan, G. J., and Davies, F. E. (2007) Heat shock protein inhibition is associated with activation of the unfolded protein response pathway in myeloma plasma cells, Blood 110, 2641-2649.
[10] Bosch, A., Eroles, P., Zaragoza, R., Vina, J. R., and Lluch, A. (2010) Triple-negative breast cancer: molecular features, pathogenesis, treatment and current lines of research, Cancer treatment reviews 36, 206-215.
[11] Petrocca, F., Altschuler, G., Tan, S. M., Mendillo, M. L., Yan, H., Jerry, D. J., Kung, A. L., Hide, W., Ince, T. A., and Lieberman, J. (2013) A genome-wide siRNA screen identifies proteasome addiction as a vulnerability of basal-like triple-negative breast cancer cells, Cancer Cell 24, 182-196.
[12] Papandreou, I., Denko, N. C., Olson, M., Van Melckebeke, H., Lust, S., Tam, A., Solow-Cordero, D. E., Bouley, D. M., Offner, F., Niwa, M., and Koong, A. C. (2011) Identification of an Ire1alpha endonuclease specific inhibitor with cytotoxic activity against human multiple myeloma, Blood 117, 1311-1314.
[13] Mimura, N., Fulciniti, M., Gorgun, G., Tai, Y. T., Cirstea, D., Santo, L., Hu, Y., Fabre, C., Minami, J., Ohguchi, H., Kiziltepe, T., Ikeda, H., Kawano, Y., French, M., Blumenthal, M., Tam, V., Kertesz, N. L., Malyankar, U. M., Hokenson, M., Pham, T., Zeng, Q., Patterson, J. B., Richardson, P. G., Munshi, N. C., and Anderson, K. C. (2012) Blockade of XBP1 splicing by inhibition of IRE1alpha is a promising therapeutic option in multiple myeloma, Blood 119, 5772-5781.
[14] Hetz, C., Chevet, E., and Harding, H. P. (2013) Targeting the unfolded protein response in disease, Nat. Rev. Drug Discov. 12, 703-719.
[15] Wang, Y., Xiao, J., Zhou, H., Yang, S., Wu, X., Jiang, C., Zhao, Y., Liang, D., Li, X., and Liang, G. (2011) A novel monocarbonyl analogue of curcumin, (1E,4E)-1,5-bis(2,3-dimethoxyphenyl)penta-1,4-dien-3-one, induced cancer cell H460 apoptosis via activation of endoplasmic reticulum stress signaling pathway, J. Med. Chem. 54, 3768-3778.
[16] Zinszner, H., Kuroda, M., Wang, X., Batchvarova, N., Lightfoot, R. T., Remotti, H., Stevens, J. L., and Ron, D. (1998) CHOP is implicated in programmed cell death in response to impaired function of the endoplasmic reticulum, Genes Dev. 12, 982-995.
[17] Marciniak, S. J., Yun, C. Y., Oyadomari, S., Novoa, I., Zhang, Y., Jungreis, R., Nagata, K., Harding, H. P., and Ron, D. (2004) CHOP induces death by promoting protein synthesis and oxidation in the stressed endoplasmic reticulum, Genes Dev. 18, 3066-3077.
[18] Ma, Y., Brewer, J. W., Diehl, J. A., and Hendershot, L. M. (2002) Two distinct stress signaling pathways converge upon the CHOP promoter during the mammalian unfolded protein response, J. Mol. Biol. 318, 1351-1365.
[19] Huber, A. L., Lebeau, J., Guillaumot, P., Petrilli, V., Malek, M., Chilloux, J., Fauvet, F., Payen, L., Kfoury, A., Renno, T., Chevet, E., and Manie, S. N. (2013) p58(IPK)-mediated attenuation of the proapoptotic PERK-CHOP pathway allows malignant progression upon low glucose, Molecular cell 49, 1049-1059.
[20] Nakagawa, H., Umemura, A., Taniguchi, K., Font-Burgada, J., Dhar, D., Ogata, H., Zhong, Z., Valasek, M. A., Seki, E., Hidalgo, J., Koike, K., Kaufman, R. J., and Karin, M. (2014) ER stress cooperates with hypernutrition to trigger TNF-dependent spontaneous HCC development, Cancer Cell 26, 331-343.
[21] Tran, K., Li, Y., Duan, H., Arora, D., Lim, H. Y., and Wang, W. (2014) Identification of Small Molecules That Protect Pancreatic beta Cells against Endoplasmic Reticulum Stress-Induced Cell Death, ACS chemical biology.
[22] Dey, S., Baird, T. D., Zhou, D., Palam, L. R., Spandau, D. F., and Wek, R. C. (2010) Both transcriptional regulation and translational control of ATF4 are central to the integrated stress response, J. Biol. Chem. 285, 33165-33174.
[23] Yoshida, H., Matsui, T., Yamamoto, A., Okada, T., and Mori, K. (2001) XBP1 mRNA is induced by ATF6 and spliced by IRE1 in response to ER stress to produce a highly active transcription factor, Cell 107, 881-891.
[24] Yamamoto, K., Sato, T., Matsui, T., Sato, M., Okada, T., Yoshida, H., Harada, A., and Mori, K. (2007) Transcriptional induction of mammalian ER quality control proteins is mediated by single or combined action of ATF6alpha and XBP1, Dev. Cell 13, 365-376.
[25] Yoshida, H., Okada, T., Haze, K., Yanagi, H., Yura, T., Negishi, M., and Mori, K. (2000) ATF6 activated by proteolysis binds in the presence of NF-Y (CBF) directly to the cis-acting element responsible for the mammalian unfolded protein response, Mol. Cell. Biol. 20, 6755-6767.
[26] De La Fuente, R., Sonawane, N. D., Arumainayagam, D., and Verkman, A. S. (2006) Small molecules with antimicrobial activity against E. coli and P. aeruginosa identified by high-throughput screening, Br. J. Pharmacol. 149, 551-559.
[27] Horrigan, S., Zong, Q, Soppet, D, Castaneda, J, Chen, B, Cibotti, R, Audoly, L, Coyle, A, Kiener, P., (2006) Compounds and methods for treating or preventing autoimmune diseases, in PCT Application WO2008144011.

1. A method of increasing the expression of mRNA level of the endogenous CHOP gene in a cell comprising the steps of:
providing one or more cells in need of increased mRNA express of a CHOP gene; and
administering an effective amount of an N-phenyl-5-nitrofuran-2-carboxamide composition to the one or more cells, wherein the N-phenyl-5-nitrofuran-2-carboxamide composition increases CHOP gene expression.

2. The method of claim 1, wherein the N-phenyl-5-nitrofuran-2-carboxamide composition activates an eukaryotic initiation factor-2α (eIF2α)-activating transcription factor 4 (ATF4) pathway to induce CHOP expression.

3. A method of activating a PERK-eIF2α-ATF4 branch of an unfolded protein response expression in a cell comprising the steps of:
providing one or more cells in need of increasing PERK-eIF2α-ATF4 branch of an unfolded protein response expression;
administering an effective amount of an N-phenyl-5-nitrofuran-2-carboxamide composition to the one or more cells; and
increasing PERK-eIF2α-ATF4 branch of an unfolded protein response expression in the one or more cells.

4. A method of inducing apoptosis in cells comprising the steps of:
providing one or more cells;
administering an effective amount of an N-phenyl-5-nitrofuran-2-carboxamide composition to the one or more cells; and
increasing the expression of mRNA of a CHOP gene to increase apoptosis in the one or more cells.

5. A method of treating one or more cancer cells comprising the steps of:
providing one or more cancer cells; and
administering an effective amount of an N-phenyl-5-nitrofuran-2-carboxamide composition, wherein the N-phenyl-5-nitrofuran-2-carboxamide composition increases the mRNA level of a CHOP gene to increase apoptosis in the one or more cancer cells to treat the one or more cancer cells.

6. The method of claim 5, wherein the one or more cancer cells are triple negative breast cancer cells.

7. The method of any of claims 1 to 6, further comprising administering one or more cytotoxic agents, chemotherapeutic agents, metal complexes, vaccines, immunotherapy agents or a combination thereof.

8. The method of any of claims 1 to 6, wherein N-phenyl-5-nitrofuran-2-carboxamide composition increases expression of a CHOP protein.

9. The method of any of claims 1 to 6, wherein N-phenyl-5-nitrofuran-2-carboxamide composition increases activity of a CHOP protein.

10. The method of any of claims 1 to 6, wherein the N-phenyl-5-nitrofuran-2-carboxamide composition has the formula: 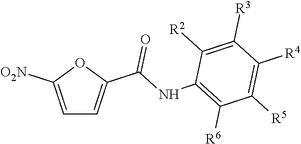
wherein R4 is a methyl, an ethyl, Cl, Br, I or F; and R2, R3, R5, and R6 are Hydrogens;
R4 is a methyl; and R2, R3, R5, and R6 are Hydrogens;
R4 is a ethyl; and R2, R3, R5, and R6 are Hydrogens;
R4 is a Cl; and R2, R3, R5, and R6 are Hydrogens;
R3 is a meOCF₃ group; and R2, R4, R5, and R6 are Hydrogens;
R2 and R5 are Cl, Br, I, or F; and R3, R4, and R6 are Hydrogens;
R2 and R5 are methyls or ethyls; R4 is a Br, Cl, I or F;, and R3, and R6 are Hydrogens;
R2 is a Cl, Br, I or F; R5 is a CF₃; and R3, R4, and R6 are Hydrogens;
R4 is a morpholine; R3 is a hydrogen; and R2, R5, and R6 are hydrogens;
R4 is a morpholine; R3 is a Cl, Br, I or F; and R2, R5, and R6 are hydrogens;
R4 is a piperidine; R3 is a Cl, Br, I or F; and R2, R5, and R6 are hydrogens;
R4 is a 4 methyl piperidine; R3 is a hydrogen; and R2, R5, and R6 are hydrogens; or
R4 is a piperazine; R3 is a hydrogen; and R2, R5, and R6 are hydrogens.

11. The pharmaceutical composition comprising a pharmaceutically acceptable diluent or carrier and a therapeutically effective amount of a N-phenyl-5-nitrofuran-2-carboxamide composition has the formula: 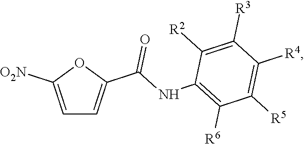
wherein the N-phenyl-5-nitrofuran-2-carboxamide composition increases the expression level of a CHOP gene mRNA.

12. The pharmaceutical composition of claim 11, wherein
R4 is a methyl, an ethyl, Cl, Br, I or F; and R2, R3, R5, and R6 are Hydrogens;
R4 is a methyl; and R2, R3, R5, and R6 are Hydrogens;
R4 is a ethyl; and R2, R3, R5, and R6 are Hydrogens;
R4 is a Cl; and R2, R3, R5, and R6 are Hydrogens;
R3 is a meOCF₃ group; and R2, R4, R5, and R6 are Hydrogens;
R2 and R5 are Cl, Br, I, or F; and R3, R4, and R6 are Hydrogens;
R2 and R5 are methyls or ethyls; R4 is a Br, Cl, I or F; and R3, and R6 are Hydrogens;
R2 is a Cl, Br, I or F; R5 is a CF₃; and R3, R4, and R6 are Hydrogens;
R4 is a morpholine; R3 is a hydrogen; and R2, R5, and R6 are hydrogens;
R4 is a morpholine; R3 is a Cl, Br, I or F; and R2, R5, and R6 are hydrogens;
R4 is a piperidine; R3 is a Cl, Br, I or F; and R2, R5, and R6 are hydrogens;
R4 is a 4 methyl piperidine; R3 is a hydrogen; and R2, R5, and R6 are hydrogens; or
R4 is a piperazine; R3 is a hydrogen; and R2, R5, and R6 are hydrogens.

13. The therapeutic composition of claim 11 or 12, further comprising a targeting molecule that binds to a cell or a portion of a cell.

14. The therapeutic composition of claim 13, wherein the targeting molecule is a protein, an antibody, a receptor antagonist, a receptor binding agent, a portion of a receptor binding agent, a portion of an antibody, or a combination thereof that binds to a cell or a portion of a cell.

15. The pharmaceutical composition of any of claims 11,12, 13 and 14, wherein the pharmaceutically acceptable carrier is a polymer, a liposome, peptide, an antibody, synthetic composition or a combination thereof.

16. The therapeutic composition of claim 11 or 12, further comprising one or more cytotoxic agents, chemotherapeutic agents, metal complexes, vaccines, immunotherapy agents or a combination thereof.

17. The use of a therapeutic compound having the chemical formula: 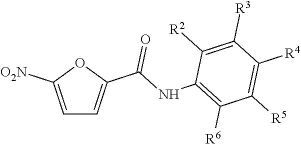
wherein R4 is a methyl, an ethyl, Cl, Br, I or F; and R2, R3, R5, and R6 are Hydrogens; R4 is a methyl; and R2, R3, R5, and R6 are Hydrogens; R4 is a ethyl; and R2, R3, R5, and R6 are Hydrogens; R4 is a Cl; and R2, R3, R5, and R6 are Hydrogens; R3 is a meOCF₃ group; and R2, R4, R5, and R6 are Hydrogens; R2 and R5 are Cl, Br, I, or F; and R3, R4, and R6 are Hydrogens; R2 and R5 are methyls or ethyls; R4 is a Br, Cl, I or F; and R3, and R6 are Hydrogens; R2 is a Cl, Br, I or F; R5 is a CF₃; and R3, R4, and R6 are Hydrogens; R4 is a morpholine; R3 is a hydrogen; and R2, R5, and R6 are hydrogens; R4 is a morpholine; R3 is a Cl, Br, I or F; and R2, R5, and R6 are hydrogens; R4 is a piperidine; R3 is a Cl, Br, I or F; and R2, R5, and R6 are hydrogens; R4 is a 4 methyl piperidine; R3 is a hydrogen; and R2, R5, and R6 are hydrogens; or R4 is a piperazine; R3 is a hydrogen; and R2, R5, and R6 are hydrogens, or a pharmaceutically acceptable salt thereof, and a pharmaceutically acceptable diluent or carrier for increasing the expression level of a CHOP gene mRNA in a cell.

18. The use of claim 17 wherein the increase in the expression level of a CHOP gene mRNA results in apoptosis of the cell.

19. The use of claim 17 wherein the increase in the expression level of a CHOP gene mRNA results in an increase in the protein expression of CHOP in the cell, increased CHOP protein activity or both.

20. The use of claim 17 wherein the therapeutic compound is used as a primary therapeutic, a secondary therapeutic or as a co-therapeutic.
